(19) United States
(12) Patent Application Publication
(10) Pub. No.: US 2025/0275715 A1
(43) Pub. Date: Sep. 4, 2025

(54) PHYSIOLOGICAL INFORMATION PROCESSING APPARATUS, PHYSIOLOGICAL INFORMATION PROCESSING METHOD, AND NON-TRANSITORY COMPUTER READABLE STORAGE MEDIUM

(71) Applicant: NIHON KOHDEN CORPORATION, Tokyo (JP)

(72) Inventors: Tetsuri ARIYAMA, Saitama (JP); Minoru MATSUSHIMA, Saitama (JP); Hideki OCHIAI, Saitama (JP)

(21) Appl. No.: 19/058,215

(22) Filed: Feb. 20, 2025

(30) Foreign Application Priority Data
Mar. 4, 2024 (JP) 2024-032496

(51) Int. Cl. A61B 5/00; A61B 5/0205; A61B 5/021; A61B 5/024; A61B 5/318

(52) U.S. Cl. CPC A61B 5/4824; A61B 5/0205; A61B 5/318; A61B 5/021; A61B 5/024

(57) ABSTRACT

A physiological information processing apparatus for evaluating a degree of pain of a patient. The physiological information processing apparatus includes a measurement unit configured to measure physiological information on the patient, a memory configured to store reference physiological information, a processor configured to compare the physiological information with the reference physiological information, and calculate a pain score of the patient, based on a comparison result between the physiological information and the reference physiological information. The processor is further configured to calculate, for each of the physiological information and the reference physiological information, at least one common physiological parameter among a heart rate, a QT interval, a pulse wave propagation time, a perfusion index, and an RR interval, and compare the physiological parameter of the physiological information with the physiological parameter of the reference physiological information.

### CROSS-REFERENCE TO RELATED APPLICATIONS

This application is based on and claims priority under 35 USC 119 from Japanese Patent Application No. 2024-032496 filed on Mar. 4, 2024, the entire content of which is incorporated herein by reference.

### TECHNICAL FIELD

The presently disclosed subject matter relates to a physiological information processing apparatus, a physiological information processing method, and a non-transitory computer readable storage medium storing a program.

### BACKGROUND ART

Generally, general anesthesia consists of three elements: sedation, analgesia, and muscle relaxation. In order to manage patient safety and improve prognosis during a perioperative period, it is necessary to properly manage these three elements under general anesthesia. There is a dedicated monitoring device for sedation and muscle relaxation. However, no monitoring device for analgesia can withstand clinical use, and no pain evaluation index exists for use during surgery.

For example, JP6934251B discloses a monitoring device that calculates a unique index nociceptive response or nociceptive reaction (NR) value from measured values of a heart rate, a systolic blood pressure, and a blood flow index. Further, a visual analogue scale (VAS) and a numerical rating scale (NRS) are generally known as pain evaluation indices.

However, in the monitoring device disclosed in JP6934251B, since explanatory variables for evaluating pain are not sufficient, pain of a patient cannot be evaluated with high accuracy. Further, pain evaluation indices such as VAS and NRS are based on a subjective evaluation of a patient, and therefore cannot be used for a patient who is not conscious, for example, a patient who is subjected to general anesthesia.

### SUMMARY OF INVENTION

Aspect of non-limiting embodiments of the present disclosure relates to provide a physiological information processing apparatus, a physiological information processing method, and a program capable of evaluating a degree of pain of a patient with high accuracy.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided a physiological information processing apparatus for evaluating a degree of pain of a patient, the physiological information processing apparatus including:
a measurement unit configured to measure physiological information on the patient;
a memory configured to store reference physiological information;
a processor configured to:
   compare the physiological information with the reference physiological information; and
   calculate a pain score of the patient, based on a comparison result between the physiological information and the reference physiological information,
in which the processor is further configured to:
   calculate, for each of the physiological information and the reference physiological information, at least one common physiological parameter among a heart rate, a QT interval, a pulse wave propagation time, a perfusion index, and an RR interval; and
   compare the physiological parameter of the physiological information with the physiological parameter of the reference physiological information.

According to an aspect of the present disclosure, there is provided a physiological information processing method for evaluating a degree of pain of a patient, the physiological information processing method including:
measuring physiological information on the patient;
comparing the physiological information with reference physiological information stored in a memory; and
calculating a pain score of the patient, based on a comparison result of the comparing,
in which the comparing includes:
   calculating, for each of the physiological information and the reference physiological information, at least one common physiological parameter among a heart rate, a QT interval, a pulse wave propagation time, a perfusion index, and an RR interval; and
   comparing the physiological parameter of the physiological information with the physiological parameter of the reference physiological information.

According to an aspect of the present disclosure, there is provided a non-transitory computer readable storage medium storing a program for causing a computer to execute processing including:
measuring physiological information on a patient;
comparing the physiological information with reference physiological information stored in a memory; and
calculating a pain score of the patient, based on a comparison result of the comparing,
in which the comparison step includes:
   calculating, for each of the physiological information and the reference physiological information, at least one common physiological parameter among a heart rate, a QT interval, a pulse wave propagation time, a perfusion index, and an RR interval; and
   comparing the physiological parameter of the physiological information with the physiological parameter of the reference physiological information.

### BRIEF DESCRIPTION OF DRAWINGS

Exemplary embodiment(s) of the present invention will be described in detail based on the following figures, wherein:

FIG. 1 is a block diagram illustrating a configuration of a physiological information measurement device according to a first embodiment of the presently disclosed subject matter;

FIG. 2 is a flowchart of pain score calculation processing according to the first embodiment of the presently disclosed subject matter;

FIG. 3 is a schematic diagram illustrating a similarity between physiological information and reference physiological information, and a pain score;

FIG. 4 is a graph illustrating a method of determining presence or absence of pain based on the similarity between the physiological information and the reference physiological information;

FIG. 5 is a block diagram illustrating a configuration of a physiological information measurement device according to a second embodiment of the presently disclosed subject matter; and

FIG. 6 is a flowchart of pain score calculation processing according to the second embodiment of the presently disclosed subject matter.

### DESCRIPTION OF EMBODIMENTS

Hereinafter, embodiments of the presently disclosed subject matter will be described with reference to the drawings. Members having the same reference numerals as those already described in the description of the embodiment will not be described for convenience of description. Further, for convenience of description, dimensions of each member illustrated in the drawings may be different from actual dimensions of each member.

### First Embodiment

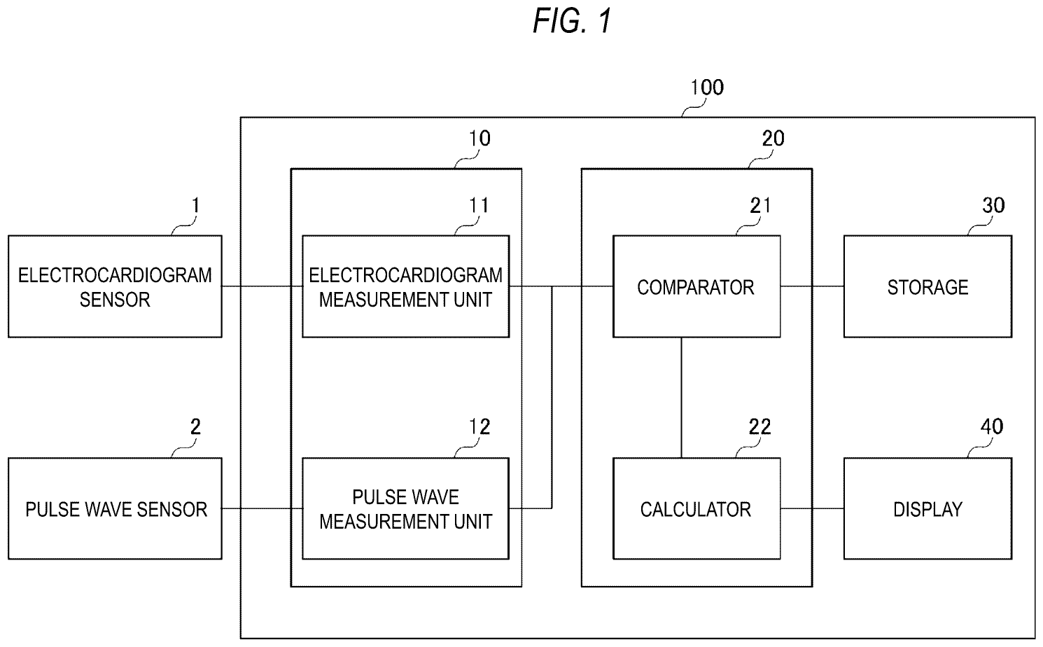

Hereinafter, a physiological information measurement device 100 according to a first embodiment will be described with reference to FIGS. 1 to 4.

FIG. 1 is a block diagram illustrating a configuration of the physiological information measurement device 100 according to the first embodiment of the presently disclosed subject matter. The physiological information measurement device 100 includes a measurement unit 10, a processor 20, a storage device 30, and a display 40. The measurement unit 10 includes an electrocardiogram measurement unit 11 and a pulse wave measurement unit 12. The processor 20 includes a comparator 21 and a calculator 22. The electrocardiogram measurement unit 11 is configured to be connected to an electrocardiogram sensor 1. The pulse wave measurement unit 12 is configured to be connected to a pulse wave sensor 2. The physiological information measurement device 100 may include one or both of the electrocardiogram sensor 1 and the pulse wave sensor 2 therein. Further, the physiological information measurement device 100 may be a medical dedicated apparatus for displaying physiological information on a patient (for example, a patient monitor), or may be, for example, a personal computer, a workstation, a smartphone, a tablet, or a wearable device worn by a medical worker.

The physiological information measurement device 100 may include a central processing unit (CPU), a read only memory (ROM), a random access memory (RAM), a hard disk drive (HDD), and the like. The CPU may function as the measurement unit 10 including the electrocardiogram measurement unit 11 and the pulse wave measurement unit 12. The CPU may function as the processor 20 including the comparator 21 and the calculator 22.

The electrocardiogram sensor 1 is configured to detect electrocardiogram data indicating an electrocardiogram waveform of a patient. The pulse wave sensor 2 (for example, a pulse oximeter) is configured to detect pulse wave data indicating a pulse wave of the patient. The electrocardiogram measurement unit 11 is configured to obtain the electrocardiogram data of the patient by controlling the electrocardiogram sensor 1. The pulse wave measurement unit 12 is configured to obtain the pulse wave data of the patient by controlling the pulse wave sensor 2. Hereinafter, the obtained physiological information such as the electrocardiogram data and the pulse wave data is referred to as physiological information BI.

The storage device 30 is, for example, a flash memory, and is configured to store programs and various data. Further, the storage device 30 may store physiological information such as reference electrocardiogram data and pulse wave data (hereinafter, referred to as reference physiological information BIR). Here, the reference physiological information BIR is physiological information indicating a response to pain that occurs in the patient due to, for example, surgical incision, tracheal intubation, tracheal injection, tracheal extubation, skin incision, suturing, puncture, sternotomy, catheter insertion, and nerve stimulation (including electrical stimulation used in TOF monitor). Hereinafter, an occurrence of such pain in the patient is referred to as a pain event. The physiological information BI and the reference physiological information BIR may be physiological information on the same person or physiological information on different persons.

The comparator 21 is configured to compare the electrocardiogram data of the patient obtained by the electrocardiogram measurement unit 11 and the pulse wave data of the patient obtained by the pulse wave measurement unit 12 with the electrocardiogram data and the pulse wave data of the reference physiological information BIR stored in the storage device 21, respectively. A reason why the electrocardiogram data and the pulse wave data are compared here is that, in general, when a pain event occurs in the patient, there is an increase in heart rate, blood pressure, and respiration rate, and changes occur in vasoconstriction and endocrine stress reactions, causing changes in physiological information such as the electrocardiogram data and the pulse wave data. Accordingly, whether a pain event has occurred in the patient can be accurately determined. Details of a method of comparing the electrocardiogram data and the pulse wave data will be described later.

The calculator 22 is configured to calculate a pain score PS of the patient based on a comparison result of the physiological information BI and the reference physiological information BIR by the comparator 21. The pain score PS indicates a degree of pain when a pain event occurs in the patient. A method of calculating the pain score PS will be described in detail later.

The display 40 includes, for example, a liquid crystal panel and an organic EL, and is configured to display the pain score PS of the patient calculated by the calculator 22 or the like to the user.

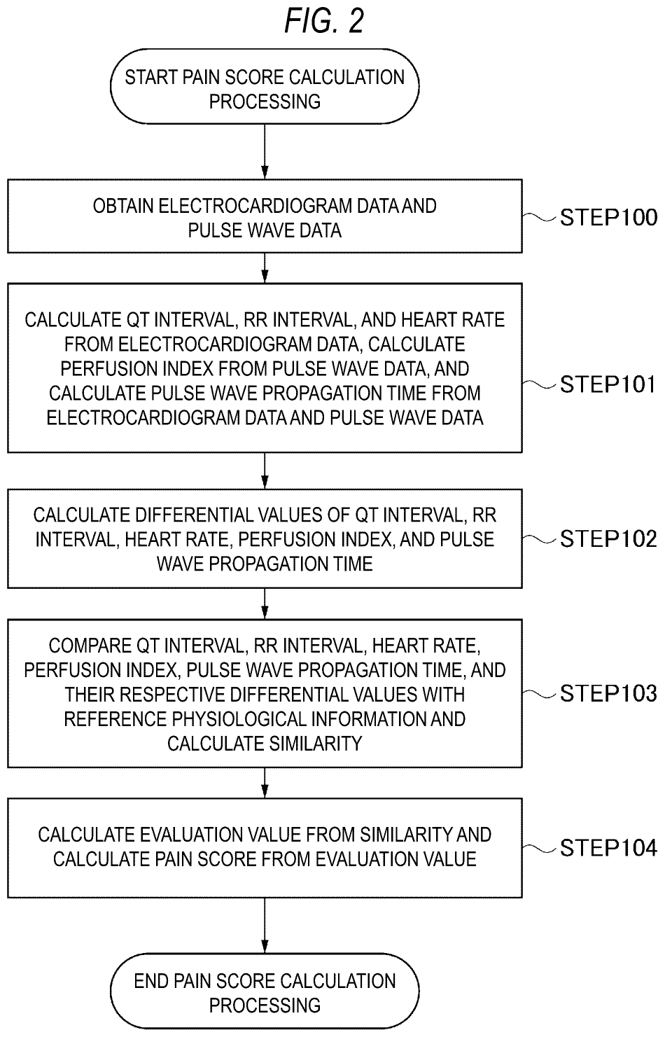

Next, a physiological information measurement method for calculating the pain score PS of the patient will be described with reference to FIG. 2. FIG. 2 is a flowchart of pain score calculation processing according to the first embodiment of the presently disclosed subject matter.

First, the electrocardiogram measurement unit 11 obtains the electrocardiogram data that is the physiological information on the patient, and the pulse wave measurement unit 12 obtains the pulse wave data that is the physiological information on the patient (STEP 100). Here, STEP 100 is referred to as a measurement step. Next, the comparator 21 calculates a QT interval, an RR interval, and a heart rate from the electrocardiogram data, calculates a perfusion index from the pulse wave data, and calculates a pulse wave propagation time from the electrocardiogram data and the pulse wave data (STEP 101). Further, the comparator 21 calculates differential values of the QT interval, the RR interval, the heart rate, the perfusion index, and the pulse wave propagation time (STEP 102). Here, the QT interval, the RR interval, the heart rate, the perfusion index, the pulse wave propagation time, and their respective differential values are collectively referred to as physiological parameters. STEPS 101 and 102 are collectively referred to as a physiological parameter calculation step. Next, the comparator 21 compares a physiological parameter of the physiological information BI with a physiological parameter of the reference physiological information BIR that is common to the physiological parameter, and calculates a similarity S (STEP 103). Here, the comparison is referred to as a physiological parameter comparison step. Further, the calculator 22 calculates an evaluation value V from the similarity S and calculates a pain score PS from the evaluation value V (STEP 104). Here, STEP 104 is referred to as a pain score calculation step.

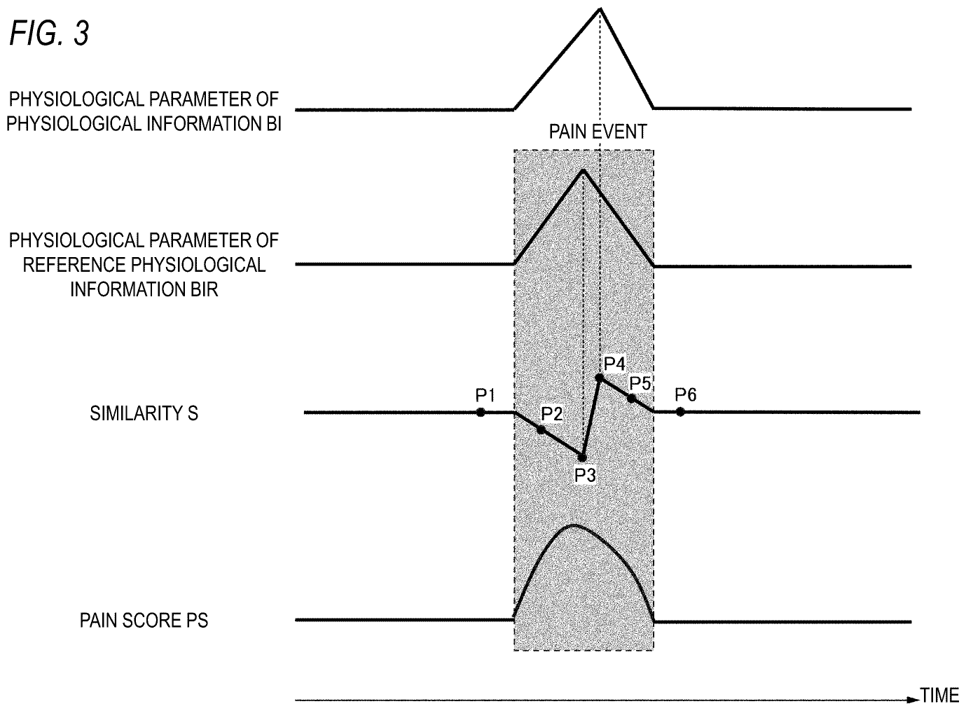

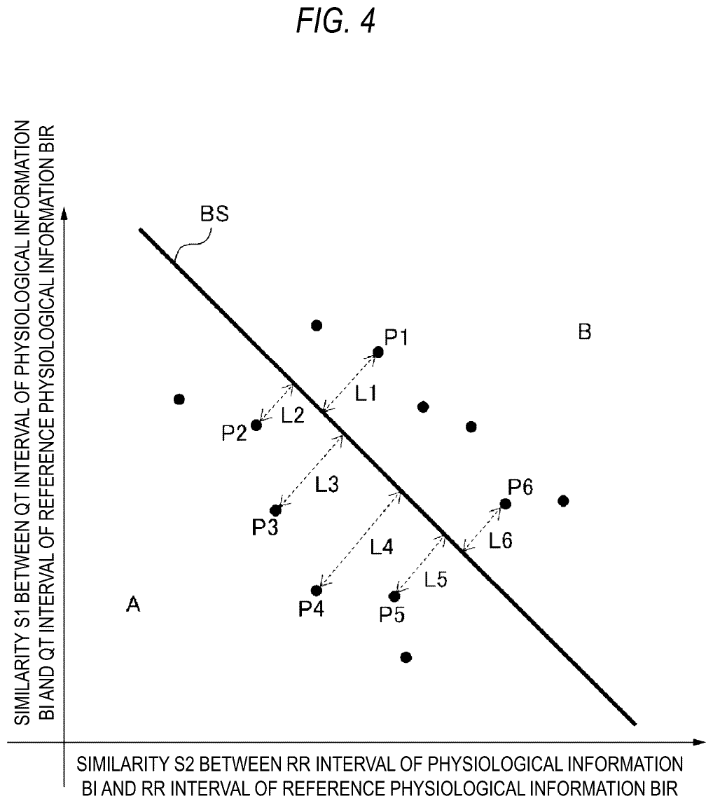

Next, a method of comparing the physiological parameter of the physiological information BI and the physiological parameter of the reference physiological information BIR by the comparator 21 and a method of calculating the pain score PS by the calculator 22 will be described in detail with reference to FIGS. 3 and 4. FIG. 3 is a schematic diagram illustrating a similarity between the physiological information and the reference physiological information, and a pain score. FIG. 4 is a graph illustrating a method of determining presence or absence of pain based on the similarity between the physiological information and the reference physiological information.

As illustrated in FIG. 3, the comparator 21 is configured to calculate a similarity S for each plot including points P1 to P6 on a timeline, for example, from a difference (Euclidean distance) between the physiological parameter of the physiological information BI and the physiological parameter of the reference physiological information BIR that is common to the physiological parameter. In the example illustrated in FIG. 3, since the reference physiological information BIR and the physiological information BI have substantially the same value at points P1 and P6, the similarity S is substantially 0. Since the physiological information BI has a smaller value than the reference physiological information BIR at points P2 and P3, the similarity S has a negative value. Since the physiological information BI has a larger value than the reference physiological information BIR at points P4 and P5, the similarity S has a positive value. In the example of FIG. 3, the similarity S is calculated from the Euclidean distance of each physiological parameter. However, the similarity S may also be calculated from cosine similarity, dynamic time warping (DTW), or cross-correlation function (CCF) of each physiological parameter.

Next, the comparator 21 is configured to calculate, for each plot including points P1 to P6 on the timeline, for example, a similarity S1 between the QT interval which is the physiological parameter of the physiological information BI and the QT interval which is the physiological parameter of the reference physiological information BIR, and a similarity S2 between the RR interval which is the physiological parameter of the physiological information BI and the RR interval which is the physiological parameter of the reference physiological information BIR, and maps the similarities S1 and S2 as points P1 to P6 illustrated in FIG. 4. Next, the comparator 21 is configured to use the similarities S1 and S2 as explanatory variables and classify each plot including points P1 to P6 as a pain event or not using a support vector machine (SVM). Specifically, a discriminant plane BS is set such that a minimum distance (margin) among distances from the plots including points P1 to P6 is maximized. In the example of FIG. 4, a distance L2 from point P2 and a distance L6 from point P6 are margins. The plots including P2 to 5 in a lower left region A from the discriminant plane BS are classified as pain events, and the plots including P1 and 6 in an upper right region B are classified as normal events in which no pain event occurs. Further, the comparator 21 is configured to calculate a distance from each plot included in the region A to the discriminant plane BS, and set the distance as an evaluation value V. For example, an evaluation value V2 of point P2 is the distance L2. Further, the calculator 22 is configured to calculate a pain score PS illustrated in a lower portion in FIG. 3 by normalizing the evaluation value V of each plot included in the region A in a range of, for example, 0 to 100. When the pain score PS is 0, it indicates a state in which there is no pain, and when the pain score PS is 100, it indicates a state in which the pain is greatest.

In the present embodiment, SVM is used as the method of determining the presence or absence of pain based on the similarity between the physiological information and the reference physiological information, and a logistic regression, a neural network, a decision tree, or a random forest may also be used to determine the presence or absence of pain.

In this way, by comparing the physiological information BI with the reference physiological information BIR and calculating the pain score PS, pain occurring in a patient can be evaluated with high accuracy even for a patient who is not conscious.

### Second Embodiment

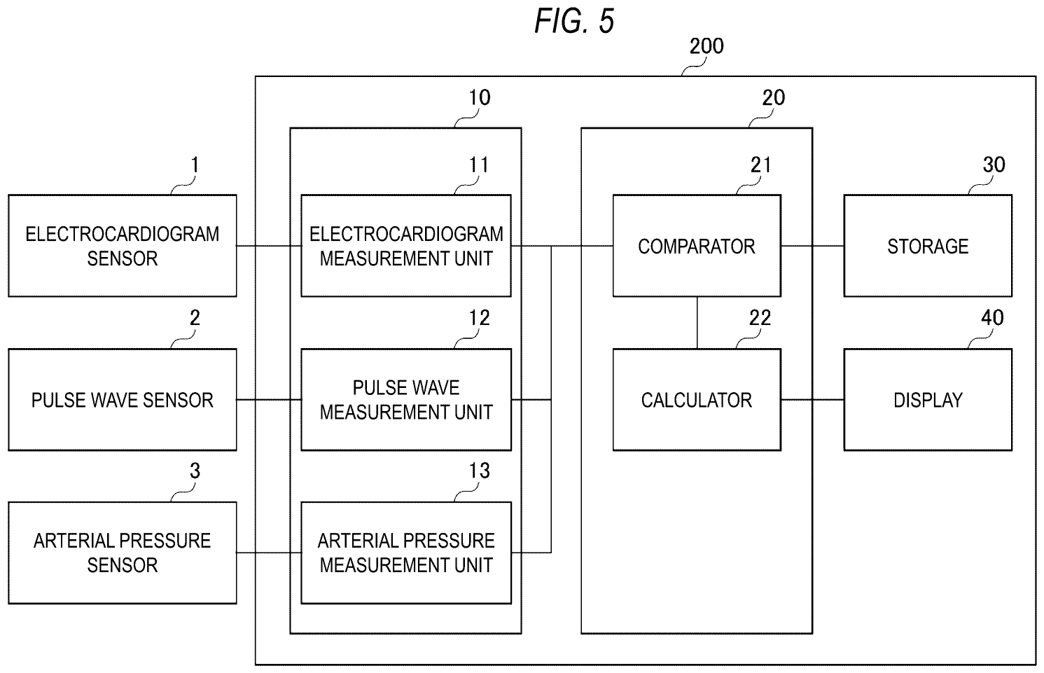

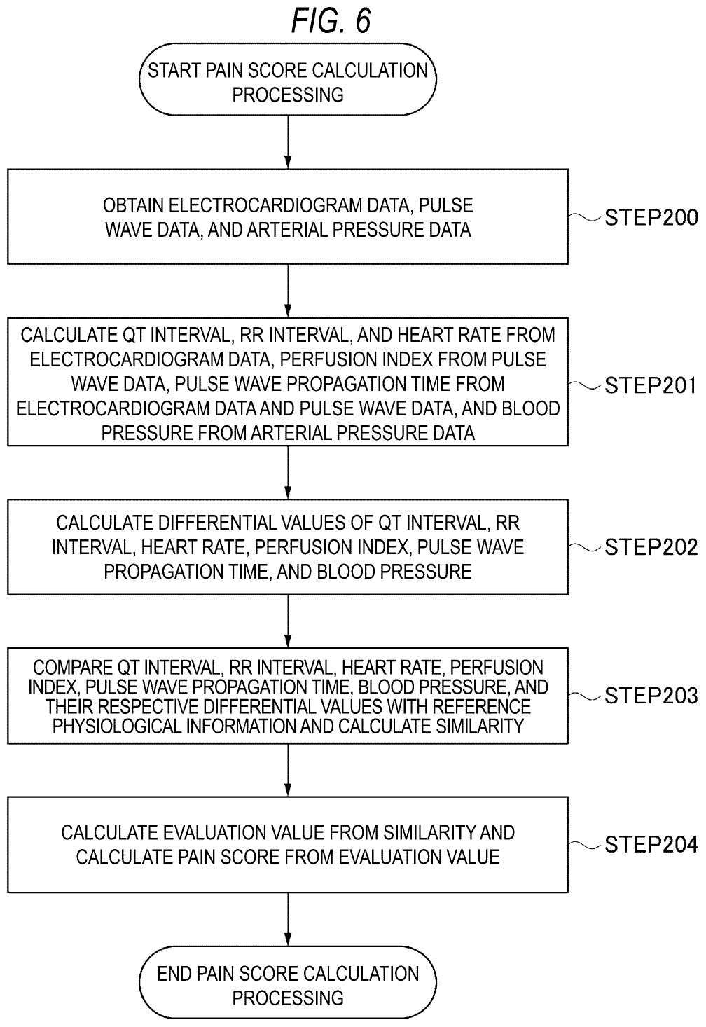

Hereinafter, a physiological information measurement device 200 according to a second embodiment will be described with reference to FIGS. 5 and 6.

FIG. 5 is a block diagram illustrating a configuration of a physiological information measurement device 200 according to the second embodiment of the presently disclosed subject matter. Hereinafter, only differences from the configuration of the physiological information measurement device 100 according to the first embodiment of the presently disclosed subject matter illustrated in FIG. 1 will be described.

The physiological information measurement device 200 includes the measurement unit 10, the processor 20, the storage device 30, and the display 40. The measurement unit 10 includes an arterial pressure measurement unit 13 in addition to the electrocardiogram measurement unit 11 and the pulse wave measurement unit 12. The arterial pressure measurement unit 13 is configured to be connected to an arterial pressure sensor 3. The arterial pressure sensor 3 is configured to detect arterial pressure data of the patient. The physiological information measurement device 200 may include the arterial pressure sensor 3 therein.

The physiological information measurement device 200 may include a central processing unit (CPU), a read only memory (ROM), a random access memory (RAM), a hard disk drive (HDD), and the like. The CPU may function as the measurement unit 10 including the arterial pressure measurement unit 13. The CPU may function as the processor 20.

The comparator 21 is configured to compare the electrocardiogram data of the patient obtained by the electrocardiogram measurement unit 11, the pulse wave data of the patient obtained by the pulse wave measurement unit 12, and arterial pressure data obtained by the arterial pressure measurement unit 13 (hereinafter, collectively referred to as physiological information BI) with the electrocardiogram data, the pulse wave data, and arterial pressure data stored in the storage device 21 (hereinafter, collectively referred to as reference physiological information BIR).

Next, a flow of a physiological information measurement method for calculating the pain score PS of the patient will be described with reference to FIG. 6. FIG. 6 is a flowchart of pain score calculation processing according to the second embodiment of the presently disclosed subject matter.

First, the electrocardiogram measurement unit 11 obtains the electrocardiogram data that is the physiological information on the patient, the pulse wave measurement unit 12 obtains the pulse wave data that is physiological information on the patient, and the arterial pressure measurement unit 13 obtains the arterial pressure data that is the physiological information on the patient (STEP 200). Next, the comparator 21 calculates a QT interval, an RR interval, and a heart rate from the electrocardiogram data, a perfusion index from the pulse wave data, a pulse wave propagation time from the electrocardiogram data and the pulse wave data, and a blood pressure from the arterial pressure data (STEP 201). The comparator 21 calculates differential values of the QT interval, the RR interval, the heart rate, the perfusion index, the pulse wave propagation time, and the blood pressure (STEP 202). Here, the QT interval, the RR interval, the heart rate, the perfusion index, the pulse wave propagation time, the blood pressure, and their respective differential values are collectively referred to as physiological parameters. Next, the comparator 21 compares a physiological parameter of the physiological information BI with a physiological parameter of the reference physiological information BIR that is common to the physiological parameter, and calculates a similarity S (STEP 203). Further, the calculator 22 calculates an evaluation value V from the similarity S and calculates a pain score PS from the evaluation value V (STEP 204).

A method of comparing the physiological parameter of the physiological information BI and the physiological parameter of the reference physiological information BIR by the comparator 21 and a method of calculating the pain score PS by the calculator 22 are the same as the comparing method and the calculating method in the first embodiment illustrated in FIGS. 3 and 4, and thus description thereof will be omitted.

As described above, by calculating the pain score PS by comparing the physiological information BI and the reference physiological information BIR, with the blood pressure included in the physiological parameters in addition to the QT interval, the RR interval, the heart rate, the perfusion index, and the pulse wave propagation time, pain occurring in a patient can be evaluated with high accuracy even for a patient who is not conscious.

The foregoing description of the exemplary embodiments of the present invention has been provided for the purposes of illustration and description. It is not intended to be exhaustive or to limit the invention to the precise forms disclosed. Obviously, many modifications and variations will be apparent to practitioners skilled in the art. The embodiments were chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

For example, in the physiological information measurement devices 100, 200 according to the embodiments of the presently disclosed subject matter, the comparator 21 may calculate a variation in the RR interval (for example, standard deviation, variance, or quartile deviation) and heart rate variability from the electrocardiogram data, calculate differential values of the variation in the RR interval and the heart rate variability, include the variation in the RR interval, the heart rate variability, and their respective differential values in the physiological parameters, and use the physiological parameters to calculate the pain score PS. A variation in a pain evaluation can be prevented by including the variation in the RR interval in the physiological parameters, and the accuracy of the pain evaluation can be improved by including the heart rate variability in the physiological parameters.

Further, in the physiological information measurement device 100 according to the embodiment of the presently disclosed subject matter, the similarity S is calculated using the QT interval, the RR interval, the heart rate, the perfusion index, the pulse wave propagation time, and their respective differential values as the physiological parameters. In the physiological information measurement device 200 according to the embodiment of the presently disclosed subject matter, the similarity S is calculated using the QT interval, the RR interval, the heart rate, the perfusion index, the pulse wave propagation time, the blood pressure, and their respective differential values as the physiological parameters. Here, the above parameters may be converted by discrete Fourier transformation (DFT) to obtain values as the physiological parameters, or the multiple parameters may be reduced in dimension by piecewise aggregate approximation (PAA) or symbolic aggregate approximation (SAX) to obtain values as the physiological parameters.

Further, the physiological information measurement devices 100, 200 according to the embodiments can be implemented as computer programs that operate in the physiological information measurement devices 100, 200. That is, the physiological information measurement devices 100, 200 each can include a processor such as a CPU and a memory.

The program is stored in a non-transitory computer-readable medium and can be read by a computer. Examples of the non-transitory computer-readable medium include a magnetic recording medium, a magneto-optical recording medium, a CD-ROM, a CD-R, a CD-R/W, and a semi-conductor memory (including an EPROM and a flash ROM). The program may be read by a computer using various types of temporary computer-readable media. Examples of the temporary computer-readable medium include an electric signal, an optical signal, and an electromagnetic wave. The temporary computer-readable medium can supply a program to the computer via a wired communication path such as an electric wire and an optical fiber or a wireless communication path.

What is claimed is:

1. A physiological information processing apparatus for evaluating a degree of pain of a patient, the physiological information processing apparatus comprising:
a measurement unit configured to measure physiological information on the patient;
a memory configured to store reference physiological information;
a processor configured to:
compare the physiological information with the reference physiological information; and
calculate a pain score of the patient, based on a comparison result between the physiological information and the reference physiological information,
wherein the processor is further configured to:
calculate, for each of the physiological information and the reference physiological information, at least one common physiological parameter among a heart rate, a QT interval, a pulse wave propagation time, a perfusion index, and an RR interval; and
compare the physiological parameter of the physiological information with the physiological parameter of the reference physiological information.

2. The physiological information processing apparatus according to claim 1,
wherein the processor is further configured to compare the physiological parameter of the physiological information with the physiological parameter of the reference physiological information, based on a similarity of the physiological parameter of the physiological information and the physiological parameter of the reference physiological information.

3. The physiological information processing apparatus according to claim 1,
wherein the pain score is an index indicating a degree of pain in a case where a pain event occurs.

4. The physiological information processing apparatus according to claim 1,
wherein the physiological information includes an electrocardiogram and a pulse wave.

5. The physiological information processing apparatus according to claim 1,
wherein the processor is configured to:
calculate an evaluation value of the physiological information for the reference physiological information, based on the comparison result; and
calculate the pain score based on the evaluation value.

6. The physiological information processing apparatus according to claim 1,
wherein the physiological information includes an electrocardiogram, a pulse wave, and an arterial pressure.

7. The physiological information processing apparatus according to claim 6,
wherein the processor is configured to calculate, from the physiological information and the reference physiological information, the physiological parameter including at least one of the heart rate, the QT interval, the pulse wave propagation time, the perfusion index, the RR interval, or a blood pressure.

8. The physiological information processing apparatus according to claim 1,
wherein the processor is configured to calculate, from the physiological information and the reference physiological information, the physiological parameter including at least one of the heart rate, the QT interval, the pulse wave propagation time, the perfusion index, the RR interval, a variation in the RR interval, or heart rate variability.

9. The physiological information processing apparatus according to claim 6,
wherein the processor is configured to calculate, from the physiological information and the reference physiological information, the physiological parameter including at least one of the heart rate, the QT interval, the pulse wave propagation time, the perfusion index, the RR interval, a blood pressure, a variation in the RR interval, or heart rate variability.

10. A physiological information processing method for evaluating a degree of pain of a patient, the physiological information processing method comprising:
measuring physiological information on the patient;
comparing the physiological information with reference physiological information stored in a memory; and
calculating a pain score of the patient, based on a comparison result of the comparing,
wherein the comparing includes:
calculating, for each of the physiological information and the reference physiological information, at least one common physiological parameter among a heart rate, a QT interval, a pulse wave propagation time, a perfusion index, and an RR interval; and
comparing the physiological parameter of the physiological information with the physiological parameter of the reference physiological information.

11. A non-transitory computer readable storage medium storing a program for causing a computer to execute processing comprising:
measuring physiological information on a patient;
comparing the physiological information with reference physiological information stored in a memory; and
calculating a pain score of the patient, based on a comparison result of the comparing,
wherein the comparison step includes:
calculating, for each of the physiological information and the reference physiological information, at least one common physiological parameter among a heart rate, a QT interval, a pulse wave propagation time, a perfusion index, and an RR interval; and
comparing the physiological parameter of the physiological information with the physiological parameter of the reference physiological information.
